(19) United States
(12) Patent Application Publication
(10) Pub. No.: US 2021/0202062 A1
(43) Pub. Date: Jul. 1, 2021

(54) MULTI-AXIS MOTOR POSITION COMPENSATION IN OPHTHALMIC SURGICAL LASER SYSTEM USING DEEP LEARNING

(71) Applicant: AMO Development, LLC, Santa Ana, CA (US)

(72) Inventors: Paul Gray, San Jose, CA (US); Cynthia Villanueva, San Jose, CA (US); Guangming Dai, Fremont, CA (US)

(21) Appl. No.: 17/102,424

(22) Filed: Nov. 23, 2020

Related U.S. Application Data
(60) Provisional application No. 62/955,198, filed on Dec. 30, 2019.

(51) Int. Cl. G16H 20/40; A61F 9/008; G06N 3/08

(52) U.S. Cl. CPC G16H 20/40; A61F 9/0084; A61F 2009/00897; G06N 3/08; A61F 2009/00872; A61F 9/00836

(57) ABSTRACT

A motor position compensation method for an ophthalmic surgical laser system employs a deep artificial neural network to characterize motor following errors of the motors of the system. The artificial neural network is trained using a large number of commanded motor positions and corresponding measured actual motor positions (measured by encoders associated with the motors) as training data, to obtain a trained artificial neural network that can predict the actual motor position for any commanded motor position. Before executing a treatment scan, the original commanded motor positions calculated from the intended scan pattern are inputted to the trained artificial neural network to predict the actual motor positions, and the predicted actual motor positions are used to adjust the original commanded motor positions. The adjusted commanded motor positions are then used to perform the treatment scan, which produces an actual scan pattern that more closely match the intended scan pattern.

### CROSS-REFERENCE TO RELATED APPLICATIONS

This application claims the benefit under 35 U.S.C. § 119(e) of U.S. Provisional Patent Application No. 62/955,198, filed Dec. 30, 2019, which is incorporated herein by reference in its entirety.

### BACKGROUND OF THE INVENTION

### Field of the Invention

Embodiments of this invention relate generally to ophthalmic surgical laser systems or other motor controlled systems, and more particularly, to systems and methods implemented in an ophthalmic surgical laser system or other motor controlled system that provide motor position compensation using deep learning technology.

### Description of Related Art

Vision impairments such as myopia (near-sightedness), hyperopia and astigmatism can be corrected using eyeglasses or contact lenses. Alternatively, the cornea of the eye can be reshaped surgically to provide the needed optical correction. Eye surgery has become commonplace with some patients pursuing it as an elective procedure to avoid using contact lenses or glasses to correct refractive problems, and others pursuing it to correct adverse conditions such as cataracts. With recent developments in laser technology, laser surgery is becoming the technique of choice for ophthalmic procedures.

Different laser eye surgical systems use different types of laser beams for the various procedures and indications. These include, for instance, ultraviolet lasers, infrared lasers, and near-infrared, ultra-short pulsed lasers. Ultra-short pulsed lasers emit radiation with pulse durations as short as 10 femtoseconds and as long as 3 nanoseconds, and a wavelength between 300 nm and 3000 nm.

To form an incision in a target eye tissue, the focal spot of the pulsed laser beam is delivered to the target eye tissue by an optical scanning system, where they interact with the eye tissue to form cavities (bubbles) in the tissue. By scanning the laser focal spot according to predefined scan patterns, the overlapping or closely adjacent bubbles form incisions (cuts) having defined two-dimensional shapes. The term “scan” or “scanning” refers to the movement of the laser focal spot along a desired path or in a desired pattern.

Prior surgical approaches for reshaping the cornea include laser assisted in situ keratomileusis (“LASIK”), photorefractive keratectomy (“PRK”) and Small Incision Lens Extraction (“SmILE”). In a LASIK procedure, an ultra-short pulsed laser is used to cut a corneal flap to expose the corneal stroma for photoablation using ultraviolet beams from an excimer laser. Photoablation of the corneal stroma reshapes the cornea and corrects the refractive condition such as myopia, hyperopia, astigmatism, and the like. In a PRK procedure where no flap is created, the epithelium layer is first removed, and some stroma material is then removed by an excimer laser. The epithelium layer will grow back within a few days after the procedure. A SmILE procedure involves tissue removal using two femtosecond laser incisions that intersect to create a lenticule shaped tissue which is then extracted. The extraction of the lenticule changes the shape of the cornea and its optical power to accomplish vision correction. Lenticular extractions can be performed either with or without the creation of a corneal flap. With the flapless procedure, a refractive lenticule is created in the intact portion of the anterior cornea and removed through a small incision.

### SUMMARY

The present invention is directed to a method and related apparatus for an ophthalmic surgical laser system that substantially obviates one or more of the problems due to limitations and disadvantages of the related art.

An object of the present invention is to provide more precise motor control for the moving components of the ophthalmic surgical laser system.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a motor position compensation method implemented in a system comprising a plurality of motors each configured to drive a moving component of the system, each motor having a corresponding encoder configured to measure actual motor positions of the motor, the system further comprising a controller operatively coupled to the plurality of motors to command the motors and receive the actual motor positions, the method including: providing a recurrent neural network (RNN) model implemented in the controller; generating training data by transmitting a first plurality of motor commands to the plurality of motors to command the plurality of motors to perform a plurality of action sequences while receiving actual motor positions of the plurality of motors from the plurality of encoders, each of the first plurality of motor commands including a commanded motor position, wherein the training data includes a plurality of data points each being a pair of a commanded motor position contained in one of the first plurality of motor commands and a corresponding actual motor position; training the RNN model using the training data to produce a trained RNN model; calculating a plurality of original commanded motor positions for each motor based on an intended action sequence; inputting the original commanded motor positions to the trained RNN model to produce corresponding predicted actual motor positions; based on differences between the predicted actual motor positions and the corresponding original commanded motor positions, adjusting the plurality of original commanded motor positions to obtain a corresponding plurality of adjusted commanded motor positions; and transmitting a second plurality of motor commands to the plurality of motors to command the plurality of motors, the second plurality of motor commands including the plurality of adjusted commanded motor positions.

In another aspect, the present invention provides a motor position compensation method implemented in a system comprising a plurality of motors each configured to drive a moving component of the system, each motor having a corresponding encoder configured to measure actual motor positions of the motor, the system further comprising a controller operatively coupled to the plurality of motors to command the motors and receive the actual motor positions, the method including: providing a trained recurrent neural network (RNN) model implemented in the controller, wherein the trained RNN model has been trained using training data that has been generated by transmitting a first plurality of motor commands to the plurality of motors to command the plurality of motors to perform a plurality of action sequences while receiving actual motor positions of the plurality of motors from the plurality of encoders, each of the first plurality of motor commands including a commanded motor position, wherein the training data includes a plurality of data points each being a pair of a commanded motor position contained in one of the first plurality of motor commands and a corresponding actual motor position; calculating a plurality of original commanded motor positions for each motor based on an intended action sequence; inputting the original commanded motor positions to the trained RNN model to produce corresponding predicted actual motor positions; based on differences between the predicted actual motor positions and the corresponding original commanded motor positions, adjusting the plurality of original commanded motor positions to obtain a corresponding plurality of adjusted commanded motor positions; and transmitting a second plurality of motor commands to the plurality of motors to command the plurality of motors, the second plurality of motor commands including the plurality of adjusted commanded motor positions.

In another aspect, the present invention provides a computer program product comprising a computer usable non-transitory medium (e.g. memory or storage device) having a computer readable program code embedded therein for controlling a data processing apparatus, the computer readable program code configured to cause the data processing apparatus to execute the above method.

It is to be understood that both the foregoing general description and the following detailed description are exemplary and explanatory and are intended to provide further explanation of the invention as claimed.

### BRIEF DESCRIPTION OF THE DRAWINGS

FIG. 1A schematically illustrate an ophthalmic surgical laser system in which embodiments of the present invention may be implemented.

FIG. 1B schematically illustrate another ophthalmic surgical laser system in which embodiments of the present invention may be implemented.

FIG. 2 is a simplified diagram of a controller and other components of a ophthalmic surgical laser system which may be used to perform a laser cutting pattern reconstruction method according to embodiments of the present invention.

FIG. 3 is a flowchart illustrating a process according to an embodiment of the present invention.

### DETAILED DESCRIPTION OF PREFERRED EMBODIMENTS

Embodiments of this invention relate to systems and methods implemented in an ophthalmic surgical laser system that provide motor position compensation using deep learning technology.

### System Configuration

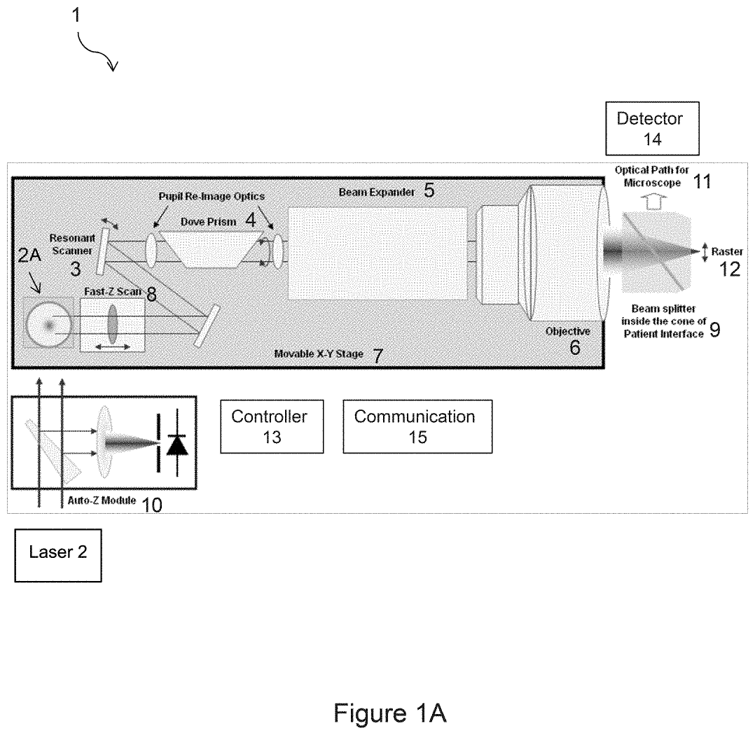

Referring to the drawings, FIG. 1A shows an ophthalmic surgical laser system 1 suitable for making an incision in a target material such as a cornea of an eye. A laser source 2, such as a femtosecond laser, provides a pulsed laser beam 2A which may be used in optical procedures to treat the eye. The system 1 further includes, but is not limited to, a high frequency scanner (such as a resonant scanner) 3 for scanning the pulsed laser beam to produce a scan line 12 of the pulsed laser beam, a scan line rotator 4 for rotating the scan line 12, a beam expander 5, an objective 6 for focusing the laser beam, an XY scan device 7 for deflecting or directing the laser beam on or within the target, a fast-Z scan device 8, a patient interface 9, an auto-Z device 10, a controller 13, and a communication module 15.

The resonant scanner 3 scans the pulsed laser beam at a high resonant frequency (e.g., thousands of Hz) to produces the scan line that extends in a lateral orientation (i.e. a direction perpendicular to the laser beam propagation direction Z) and having a desired length, for example, between 1 mm and 2 mm. The length of the scan line may be adjustable. The scan line rotator 4 may be implemented by a Dove prism, a Pechan prism, a set of mirrors, or the like, mounted on a rotating stage. By rotating the scan line rotator 4 around the Z axis, the lateral orientation of the scan line 12 is rotated, so that the scan line may be placed at any desired orientation in the XY plane (i.e., the lateral plane perpendicular to the laser beam propagation direction Z). The XY scan device 7 may be a movable XY scanning stage having the focusing objective 6 mounted thereon; the XY scan device 7 carries the objective 6 and moves it relative to the patient interface device 9, so as to move the center of the scan line 12 relative to the patient's eye in the XY directions. The fast-Z scan device 8 changes the depth (i.e. along the Z direction) of the laser focal spot location in the eye. Thus, the scan line rotator 4 modifies the lateral orientation of the scan line 12 while the moveable XY scanning stage 7 and the fast-Z scan device 8 move the center of the scan line in X, Y and Z directions. Because the scanning speed of the resonant scanner is typically much faster than the speed of the XY scanning stage and the fast-Z scan device, the scan line 12 may be referred to as a fast scan line, and the movement of the fast scan line in X, Y and Z directions may be referred to as a slow sweep.

The XY scanning stage 7 may be a motorized stage with two motors that drive its movements in the X and Y directions. Preferably, the XY scanning stage is a recoilless stage configured to reduce or eliminate mechanical vibration. The fast-Z scan device 8 may include a voice coil actuator that drives a lens in the Z direction. Movements of the lens lead to a focus depth change. The z-scan frequency may be between 50 Hz and 15,000 Hz.

The patient interface device 9 couples the patient's eye to the ophthalmic surgical laser system 1. The patient interface 9 may include a visualization beam splitter to reflect the light from the eye along an optical path 11 toward a video microscope or ocular microscope 14, to allow the eye to be imaged by an image detector of the microscope.

The auto Z module 10 measures a distal end surface of a lens of the patient interface coupled to the patient's eye and provides a depth reference for the fast-Z scan device 8 of the ophthalmic laser system. The auto Z module 10 may include, for example, a confocal detector.

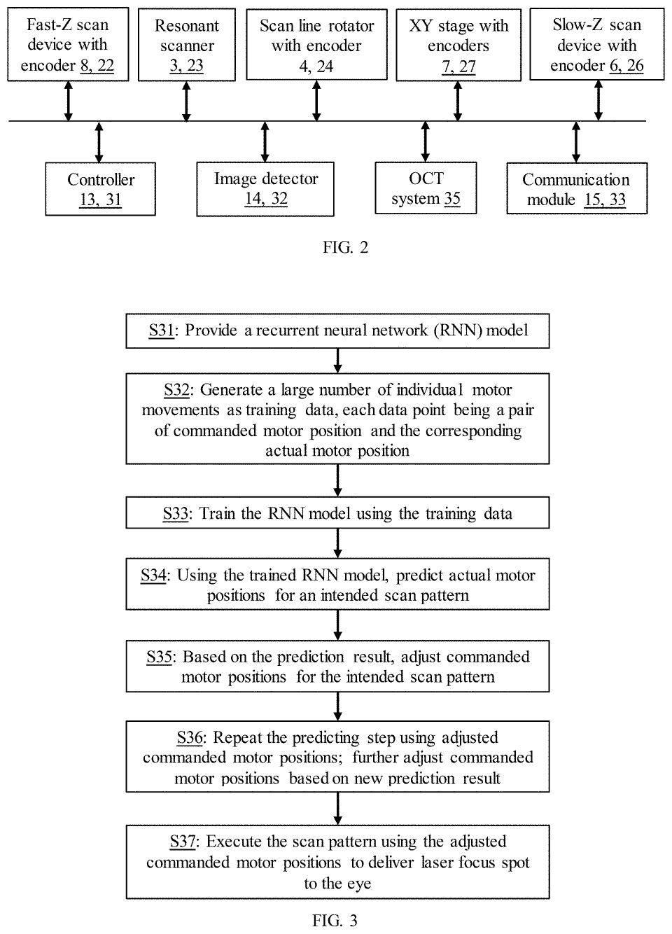

The controller 13, which may be implemented by a processor executing suitable machine-readable program code and data stored in a non-volatile memory, is operably coupled to the various components of the system 1 including the laser 2, the fast-Z scan device 8, the resonant scanner 3, the scan line rotator 4, the XY scanning stage 7, the detector 14, and the communication module 15. The controller 13 is configured to direct these components of the system to output the focal spot of the pulsed laser beam in a desired pattern in the eye so as to modify the eye. The communication module 15 provides information to the operator of the laser system 1 at the system and/or remotely via wired or wireless data connection, and may include displays, user input devices such as keyboard, mouse, joystick, etc. The ophthalmic surgical laser system may additionally include an OCT (optical coherence tomography) device (not shown in FIG. 1A, but shown in FIG. 3 as OCT 35) which may be used to measure structures of the target (e.g. eye tissues).

FIG. 1B shows an ophthalmic surgical laser system 20 suitable for making an incision in a target material such as a cornea of an eye. The system 20 includes, but is not limited to, a laser source (not shown) that generates an input pulsed laser beam 21, a fast-Z scan device 22, a resonant scanner 23 for producing a scan line 30 of the pulsed laser beam 21, a scan line rotator 24 for rotating the lateral orientation of the scan line 30, a beam expander 25, an objective with an adjustable focusing mechanism (slow-Z scanner) 26, a XY scanning stage 27 for deflecting or directing the pulsed laser beam 21 on or within the target, a patient interface 28 that may include a beam splitter, a controller 31, an image detector 32 disposed on an optical path 29 defined by the beam splitter of the patient interface, and a communication module 33. The slow-Z scanner 26 may be used to set the laser focal spot at a desired focal depth which may set the Z-baseline of the scan pattern.

One difference between the embodiment of FIG. 1B and that of FIG. 1A is that the XY scanning stage 7 in FIG. 1A carries both the objective 6 and other components including the fast-Z scan device 8, resonant scanner 3, scan line rotator 4, and beam expander 5, while the XY scanning stage 27 in FIG. 1B carries the objective 26 but not the other components mentioned above. Note that the in the system of FIG. 1A, the objective 6 may also be equipped with a slow-Z scanner (also represented by reference symbol 6).

Further details of ophthalmic surgical laser systems having the configurations shown in FIGS. 1A and 1B are described in commonly owned U.S. patent application Ser. No. 14/970,898, filed Dec. 16, 2015, entitled “Compact Ultra-Short Pulsed Laser Eye Surgery Workstation,” and Ser. No. 14/865,396, filed Sep. 25, 2015, entitled “Systems and Methods for Lenticular Laser Incision,” which are incorporated herein by reference in their entireties.

In other embodiments, an ophthalmic surgical laser system may employ other types of scanners, such as two orthogonal scanning mirrors, for scanning the laser beam in the transverse (XY) directions. Many such systems are known and their details are not described here.

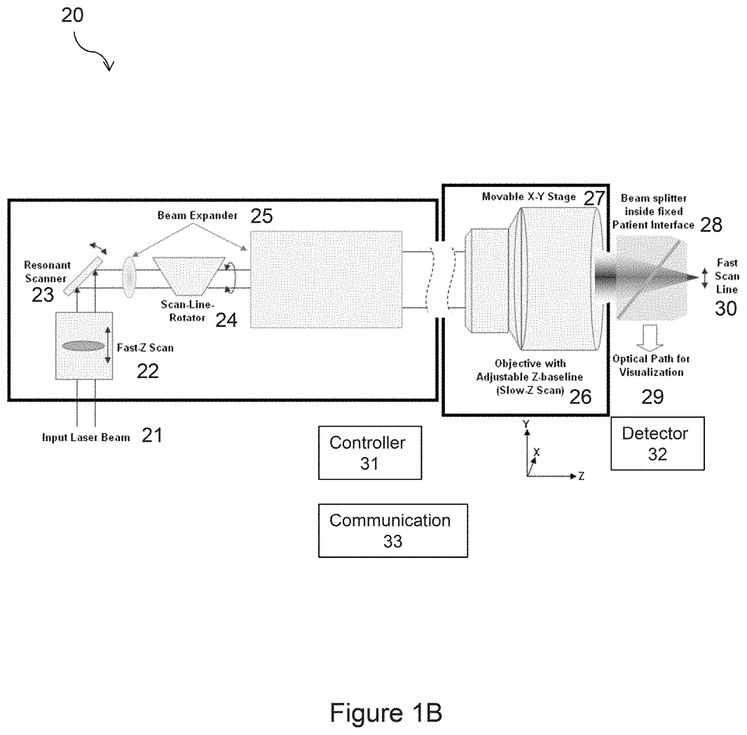

In the ophthalmic surgical laser systems shown in FIGS. 1A and 1B, the fast-Z scan device 8, 22, the resonant scanner 3, 23, the scan line rotator 4, 24, the XY scanning stage 7, 27, and slow-Z scanner 6, 26 collectively constitutes the laser beam delivery system which delivers the laser focal spot to the target. These moving components include respective actuators (e.g. linear and/or rotational actuators) which drive their movements. In these moving components, except for the resonant scanner, the actuators are equipped with respective digital encoders (position sensors) to measure and output their movements and/or positions as functions of time. The encoders are referred to as digital encoders as they output digital data; the actual mechanisms of measuring the movements and/or positions may by any suitable mechanisms such as optical, magnetic, electrical, etc. Linear and rotational actuators with integrated encoders are commercially available and may be used to implement the embodiments; their structures are known to those skilled in the art, and detailed descriptions are omitted here. The actuators and encoders of the moving components of the system of FIGS. 1A and 1B are electrically coupled to the controller 13, 31, as schematically illustrated in FIG. 2. The controller 13, 31 commands the movements of these moving components, and receives the digital outputs of their respective encoders. Note that if the data from an encoder is movement data, the controller can convert movement to position of the encoder by accumulating the movement data.

The controller controls each actuator (referred to as motor for convenience) of the beam delivery system by transmitting a series of motor commands which contain commanded motor positions and/or movements that are calculated based on an intended scan pattern. However, when the controller commands a series of motor movements during a scan, positional errors between the commanded motor positions and/or movements and the actual positions and/or movements achieved by the motor, referred to as the motor following error, may be present. The motor following error typically has a systematic component and a random component, where the random component is typically smaller than the systematic component. Motor following errors in the various moving components of the laser beam delivery system, whether systematic or random, causes the actual laser focal spot position delivered in the target to deviate from the intended positions as defined by the intended scan pattern, causing the actual achieved laser scan pattern to be less than ideal.

Reducing systematic following error proactively involves analyzing and characterizing the systematic motor following error under a variety of different motor movements of the laser beam delivery system. This process, if performed manually, is labor intensive and costly. Additionally, any change in the physical motor hardware or load on the motors can cause the characterization to become obsolete, and as a result, error correction designed based on previous characterizations may in fact add rather than reduce error.

Motor following error may also be dealt with reactively at runtime. Using a feed forward error handling technique, the system measures current error state and adjusts to the current error state in the next step.

Embodiments of the present invention provide a method and related apparatus for correcting and compensating for systematic motor following errors in the ophthalmic surgical laser system. The motor position compensation method combines proactive prediction, which compensates for the systematic following error, and real time feed forward error handling at runtime, which compensates for random following error. A deep learning technique employing an artificial neural network is used to characterize the systematic motor following errors.

More specifically, a control system according to embodiments of the present invention employs a recurrent neural network (RNN) model (step S31). RNNs are a class of artificial neural networks having multiple hidden layers of nodes, in which connections between nodes form a directed graph in a temporal sequence. RNNs, which are useful for modeling temporal data and sequential data, are generally known in the art (see, for example, the Wikipedia article Recurrent neural network, available online at https://en.wikipedia.org/wiki/Recurrent_neural_network), and RNN models are publicly available from various sources such as Tensorflow, Tensorflow-Keras, Pytorch, GitHub, the Comprehensive R Archive Network (CRAN), etc. Any suitable RNN models may be used to implement the embodiments. In one embodiment, the RNN model is a bidirectional LSTM (Long Short Term Memory) model implemented via Tensorflow with multiple fully connected layers below the bidirectional LSTM layer.

A large number (e.g. millions) of individual motor movements are generated (for example, from a number of verification and validation procedures and other parameter-tuning research activities) as training data (step S32) and fed into the RNN model to train the model to characterize motor movements (step S33). Each motor movement (a data point) in the training data is a pair of commanded motor position (as input data) and the corresponding measured actual motor position (as label data). They are either both absolute positions (i.e. to move to a certain position) or both relative positions (i.e. to move by a certain amount). The commanded motor positions are the positions defined by the motor commands issued by the controller, and the measured actual motor positions are positions measured by the encoders associated with the motors. The training data is generated by commanding the various motors of the laser beam delivery system, preferably concurrently or synchronously, to perform various scans while recording the actual motor positions. Each such scan may involve a large number of individual motor movements by multiple motors. The RNN model generates predicted actual motor positions from the commanded motor positions (input data), and is trained to minimize the differences between the predicted actual motor positions and the measured actual motor position (label data). Any suitable training method may be used to train the RNN model.

The trained RNN model may then be used to predict the actual motor positions for an intended scan pattern before executing the scan on the eye (step S34). This step includes calculating the original commanded motor positions for each motor based on the intended scan pattern, and inputting the original commanded motor positions into the trained RNN model to generate predicted actual motor positions. Based on the predicted actual motor positions, the original commanded motor positions are adjusted, e.g. by subtracting the following error (which is the differences between the predicted actual motor positions and the original commanded motor positions) (step S35). As a result, the adjusted commanded motor positions, when executed, will produce actual motor positions that more closely match the original commanded motor positions. Optionally, the predicting step may be repeated using the adjusted commanded motor positions to predict the new actual motor positions, and the adjusted commanded motor positions is further adjusted based on the differences between the predicted new actual motor positions and the original commanded motor positions (step S36), so that the further adjusted commanded motor positions, when executed, will produce actual motor positions that even more closely match the original commanded motor positions. Such further adjustments may be performed multiple times if desired. The scan pattern may then be executed on the ophthalmic surgical laser system using the final adjusted commanded motor positions to deliver the laser focal spot to the eye tissue for treatment (step S37).

The above-described motor position compensation method may be implemented in the controller of the ophthalmic surgical laser system. Alternatively, some of the steps, such as the RNN training step, may be performed on another computer system and the trained RNN model may be transferred to the controller of the ophthalmic surgical laser system.

In a specific example, four motors in the laser beam delivery system, referred to as axis 0 through axis 3 here (in this disclosure, the term “axis” refers to a degree of freedom), are chosen to perform the motor position compensation method. Axis 0, 1, and 2 are linear positional motors with units of measurement in microns, for example, the motors that control the XY scan device 7, 27 and the fast-Z scan device 8, 22 in the exemplary surgical laser system shown in FIGS. 1A and 1B. Axis 3 is a rotational motor with units in degrees, for example, the motor that controls the scan line rotator 4, 24 in the exemplary surgical laser system shown in FIGS. 1A and 1B. A bidirection LSTM with 5 fully connected layers was trained on a training dataset containing more than 40 million pairs of commanded motor positions and actual positions for each motor axis using the Tensorflow-Keras API. The scan patterns that were performed to generate the training dataset included a variety of patterns that are commonly used in actual ophthalmic procedures, such as lenticule extraction patterns (e.g. for forming a lenticule volume in the cornea to be extracted for refractive correction), flap patterns (e.g. for forming a corneal flap in a LASIK procedure), and patterns used for creating pockets, tunnels, incisions, and the like for aiding other procedures, such as inlay, etc., as well as a variety of generic scan patterns that are not commonly used for actual treatment procedures. Including these generic scan patterns in the generation of training data allows for the RNN model to be generalized for handling possible scan patterns that can be used in procedures that may be developed in the future.

A number of test scan patterns were then executed twice, once by following steps S34 (prediction) and S35 (compensation) of FIG. 3 (experimental runs), and once without any motor command compensation (control runs). The test scan patterns included ten lenticule extraction treatments and ten flap treatments, with scan parameters chosen at random. The actual motor positions during the scans were measured by the encoders associated with the motors, and the motor following errors, i.e., the difference between the actual motor positions and the commanded motor positions, were computed for each motor movement. Using the data from the ten lenticule extraction treatments, the root-mean-square value of the following errors (RMSE) was computed for each axis, for both the experimental runs and the control runs respectively. An average reduction in RMSE achieved by the experimental runs relative to the control runs, as well as a percentage reduction it represents, were computed, the results of which are shown in Tables I below.

TABLE I

| | Average reduction in RMSE | % reduction |
|---|---|---|
| Axis 0 | 10.7 μm | 71.1% |
| Axis 1 | 11.1 μm | 77.4% |
| Axis 2 | 1.1 μm | 85.5% |
| Axis 3 | 0.3 degrees | 40.7% |

Additionally, the actual measured motor positions are used to calculate the positions of the laser focal spot in the target, to reconstruct the shape of the incision. The reconstructed shapes for the experimental runs show that the average spherical equivalent error to the intended treatment scan was reduced by 0.013 diopters, a 15.9% reduction in error, as compared to the control runs.

Likewise, the reduction in RMSE for the ten flap treatments were computed, the results of which are shown in Tables II below.

TABLE II

| | Average reduction in RMSE | % reduction |
|---|---|---|
| Axis 0 | 2.2 μm | 48.6% |
| Axis 1 | 1.7 μm | 41.7% |
| Axis 2 | 0.5 μm | 48.4% |
| Axis 3 | 0.3 degrees | 27.4% |

It can be seen that the motor position compensation method described above can compensate for a significant percentage of the overall motor following error in the system.

As mentioned earlier, when executing the scan pattern, a feed forward error handling technique may be performed in real time, as a part of step S37 of FIG. 3. Feed forward error handling techniques are well known and its details are not described here.

The RNN model may be trained using training data generated on a number of individual ophthalmic surgical laser systems of the same design, and then used to perform predictions for scans to be executed on individual ophthalmic surgical laser systems of the same design. Alternatively, the RNN model may be trained using training data generated on one specific ophthalmic surgical laser system, and then used to perform predictions for scans to be executed on that system. The latter method allows the RNN model to predict systematic positional errors which are specific to one system, which may achieve better specialization in error reduction.

More generally, the motor position compensation method described above may be applied in fields outside of ophthalmic surgical laser system, or outside the medical field. The method is applicable to any field in which a controller commands a plurality of motors to perform action sequences (a laser scan being a specific example). For example, traditional CNC (computer numerical control) systems and 3D printers will likely benefit from this error compensation method.

In another aspect, the present invention provides a computer program product comprising a computer usable non-transitory medium (e.g. memory or storage device) having a computer readable program code embedded therein for controlling an ophthalmic surgical laser system, the computer readable program code configured to cause the ophthalmic surgical laser system to execute the above described motor position compensation method.

It will be apparent to those skilled in the art that various modification and variations can be made in the motor position compensation method and related apparatus of the present invention without departing from the spirit or scope of the invention. Thus, it is intended that the present invention cover modifications and variations that come within the scope of the appended claims and their equivalents.

What is claimed is:

1. A motor position compensation method implemented in a system comprising a plurality of motors each configured to drive a moving component of the system, each motor having a corresponding encoder configured to measure actual motor positions of the motor, the system further comprising a controller operatively coupled to the plurality of motors to command the motors and receive the actual motor positions, the method comprising:
providing a recurrent neural network (RNN) model implemented in the controller;
generating training data by transmitting a first plurality of motor commands to the plurality of motors to command the plurality of motors to perform a plurality of action sequences while receiving actual motor positions of the plurality of motors from the plurality of encoders, each of the first plurality of motor commands including a commanded motor position, wherein the training data includes a plurality of data points each being a pair of a commanded motor position contained in one of the first plurality of motor commands and a corresponding actual motor position;
training the RNN model using the training data to produce a trained RNN model;
calculating a plurality of original commanded motor positions for each motor based on an intended action sequence;
inputting the original commanded motor positions to the trained RNN model to produce corresponding predicted actual motor positions;
based on differences between the predicted actual motor positions and the corresponding original commanded motor positions, adjusting the plurality of original commanded motor positions to obtain a corresponding plurality of adjusted commanded motor positions; and
transmitting a second plurality of motor commands to the plurality of motors to command the plurality of motors, the second plurality of motor commands including the plurality of adjusted commanded motor positions.

2. The motor position compensation method of claim 1, wherein the RNN model is a bidirectional LSTM (Long Short Term Memory) model having a bidirectional LSTM layer multiple fully connected layers below the bidirectional LSTM layer.

3. The motor position compensation method of claim 1, wherein the system is an ophthalmic surgical laser system which further includes a laser source configured to generate a pulsed laser beam, and a laser beam delivery system configured to deliver a laser focal spot of the laser beam to a target tissue of a patient's eye, the laser beam delivery system including a plurality of optical elements each configured to interact with the laser beam and the plurality of motors each configured to move at least one of the plurality of optical elements, and wherein the intended action sequence includes scanning the laser focal spot in the patient's eye using the laser beam delivery system.

4. The motor position compensation method of claim 3, wherein in the step of generating training data, the plurality of action sequences includes scanning the laser focal spot in the patient's eye using the laser beam delivery system to form multiple incision patterns in a cornea of the eye, including one or more lenticule patterns, one or more flap patterns, one or more pocket patterns, and one or more tunnel patterns.

5. A motor position compensation method implemented in a system comprising a plurality of motors each configured to drive a moving component of the system, each motor having a corresponding encoder configured to measure actual motor positions of the motor, the system further comprising a controller operatively coupled to the plurality of motors to command the motors and receive the actual motor positions, the method comprising:
providing a trained recurrent neural network (RNN) model implemented in the controller, wherein the trained RNN model has been trained using training data that has been generated by transmitting a first plurality of motor commands to the plurality of motors to command the plurality of motors to perform a plurality of action sequences while receiving actual motor positions of the plurality of motors from the plurality of encoders, each of the first plurality of motor commands including a commanded motor position, wherein the training data includes a plurality of data points each being a pair of a commanded motor position contained in one of the first plurality of motor commands and a corresponding actual motor position;
calculating a plurality of original commanded motor positions for each motor based on an intended action sequence;
inputting the original commanded motor positions to the trained RNN model to produce corresponding predicted actual motor positions;
based on differences between the predicted actual motor positions and the corresponding original commanded motor positions, adjusting the plurality of original commanded motor positions to obtain a corresponding plurality of adjusted commanded motor positions; and
transmitting a second plurality of motor commands to the plurality of motors to command the plurality of motors, the second plurality of motor commands including the plurality of adjusted commanded motor positions.

6. The motor position compensation method of claim 5, wherein the RNN model is a bidirectional LSTM (Long Short Term Memory) model having a bidirectional LSTM layer multiple fully connected layers below the bidirectional LSTM layer.

7. The motor position compensation method of claim 5, wherein the system is an ophthalmic surgical laser system which further includes a laser source configured to generate a pulsed laser beam, and a laser beam delivery system configured to deliver a laser focal spot of the laser beam to a target tissue of a patient's eye, the laser beam delivery system including a plurality of optical elements each configured to interact with the laser beam and the plurality of motors each configured to move at least one of the plurality of optical elements, and wherein the intended action sequence includes scanning the laser focal spot in the patient's eye using the laser beam delivery system.
